(19) United States
(12) Patent Application Publication
(10) Pub. No.: US 2017/0273601 A1
(43) Pub. Date: Sep. 28, 2017

(54) SYSTEM AND METHOD FOR APPLYING BIOMECHANICAL CHARACTERIZATIONS TO PATIENT CARE

(71) Applicant: Lumo BodyTech, Inc, Mountain View, CA (US)

(72) Inventors: Chung-Che Charles Wang, Mountain View, CA (US); Andrew Robert Chang, Sunnyvale, CA (US)

(21) Appl. No.: 15/471,958

(22) Filed: Mar. 28, 2017

Related U.S. Application Data
(60) Provisional application No. 62/314,312, filed on Mar. 28, 2016.

(51) Int. Cl. A61B 5/11; A61B 5/00

(52) U.S. Cl. CPC A61B 5/1118; A61B 5/0002; A61B 5/0022; A61B 2562/0219

(57) ABSTRACT

A system and method for addressing patient health through monitoring patient movement that includes, during a treatment stage, collecting kinematic data from at least one inertial measurement unit of an activity monitoring system; generating a set of biomechanical signals from the kinematic data wherein the set of biomechanical signals characterize at least one biomechanical property; updating a mobility quality score of the subject based on the set of biomechanical signals; and delivering a health assessment.

### CROSS-REFERENCE TO RELATED APPLICATIONS

This application claims the benefit of U.S. Provisional Application No. 62/314,312, filed on 28 Mar. 2016, which is incorporated in its entirety by this reference.

### TECHNICAL FIELD

This invention relates generally to the field of health care, and more specifically to a new and useful system and method for applying biomechanical characterizations to patient care.

### BACKGROUND

The biomechanics of an individual can show considerable information, which can be useful in the treatment of an individual. However, biomechanical information is not commonly used as a primary metric of health because of the associated challenges. Rigorous understanding of the biomechanics of a patient often requires motion capture or other complicated systems, which can make it infeasible in many situations. Most often the biomechanics of an individual are visually inspected and characterized by a caregiver, nurse or physician. However, this approach is open to numerous errors and inconsistencies. Thus, there is a need in the healthcare field to create a new and useful system and method for applying biomechanical characterizations to patient care. This invention provides such a new and useful system and method.

### BRIEF DESCRIPTION OF THE FIGURES

FIG. 1 is a schematic representation of a system of a preferred embodiment;

FIG. 2 is a flowchart representation of a method of a preferred embodiment;

FIG. 3 is a flowchart representation of an implementation of the method for a treatment use case;

FIG. 4 is a schematic representation of the system and method used across multiple participants;

FIGS. 5A-5G are schematic representations of exemplary single and multi point sensing configurations;

FIG. 6 is an exemplary chart of quality score comparisons used in treatment evaluation; and

FIG. 7 is a schematic representation of configuring a sensing mode.

### DESCRIPTION OF THE EMBODIMENTS

The following description of the embodiments of the invention is not intended to limit the invention to these embodiments but rather to enable a person skilled in the art to make and use this invention.

### 1. Overview

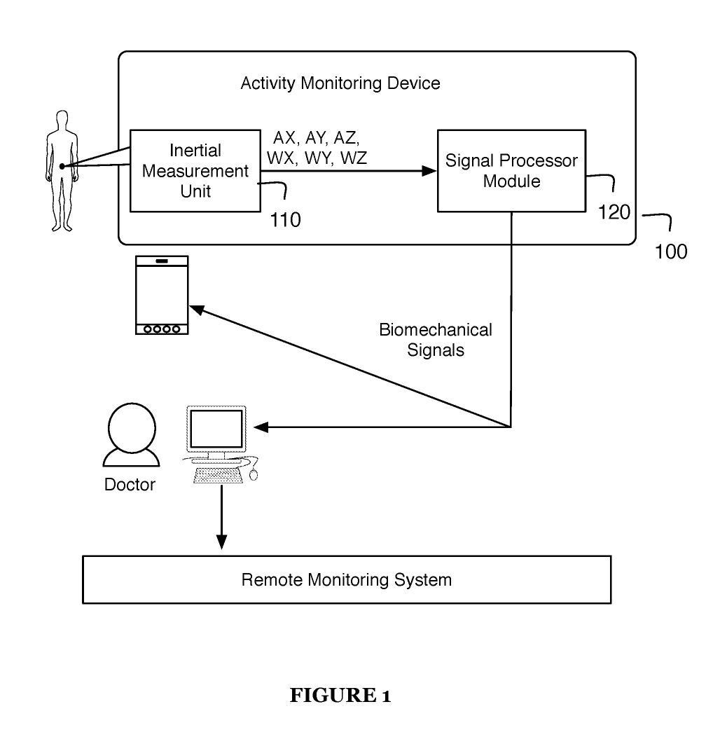

As shown in FIG. 1, a system and method for applying biomechanical characterizations to patient care of a preferred embodiment involves the transformation of collected data from an inertial measurement unit into a set of biomechanical signals. Through such biomechanical characterizations of motion and activity, the system and method can assess a motion quality of a patient, which can be used in analyzing and directing actions involved in the care for a patient. The system and method function to use kinematic sensor data in generating a detailed biomechanical characterization of motion properties. The system and method can then apply those biomechanical characterizations in various healthcare scenarios. The biomechanical characterizations of motion properties are preferably manifested through a set of biomechanical signals, which can be used in generating a report, triggering a system response, or producing any suitable tangible result. In particular, the system and method can be used in diagnosing a patient (e.g., pre-surgery analysis), in monitoring and administrating treatment (e.g., directing the dosing of pharmaceuticals, directing use of a medical device, directing physical therapy, and the like), event detection, hospital monitoring, and/or quality of care analysis. In some use cases, the system and method can be used in a single patient implementation. In other use cases, the system and method can be applied to monitoring and acting based on a plurality of distinct participants.

As a first potential benefit, the system and method can be used in translating a set of traditionally subjective judgments by a doctor into quantitative measurements. The system and method preferably generates repeatable measurements that can act as a higher resolution characterization of motion quality. Additionally, the characterization generated by the system and method can achieve greater consistency that subjective judgments made by medical staff. Furthermore, the motion quality characterization can be broken down into different biomechanical signal properties so that the motion quality of specific biomechanical actions can be individually analyzed. The motion quality analysis by the system and method will preferably yield similar results regardless of who is caring for the patient. Furthermore, the system and method can result in efficiencies during medical care and can result in faster and more accurate consultations by the medical staff.

As a related potential benefit, the system and method can enable comparative analysis. By transforming sensed motion and biomechanical properties into a motion quality measurement based on biomechanical signals, the system and method can employ comparisons between different users and at different points in time. The system and method additionally include integration with a distributed platform, and the comparative analysis can enable various scopes of analysis to be achieved such that motion quality can be judged against an individual patient (e.g., determining if a patient experienced motion quality improvement or deterioration), against a particular type of patient (e.g., evaluation of motion quality compared to a particular demographic or others with similar conditions), and/or against a healthy example.

Furthermore such quantitative measurements are achievable through a convenient and wearable device. An activity monitoring device is preferably worn or simply attached to a user. The system and method are preferably robust to different orientations during physical coupling with the patient. Such convenience can be beneficial to enable patients who may have limited mobility to use the system without assistance.

As another potential benefit, the system and method can be a substantially affordable solution compared to more complex systems that may depend on expensive medical equipment. Such economical efficiencies can enable the system and method to be more widely used in the healthcare space. As one example, it can be economically feasible to use the system and method across a wide number of patients for remote monitoring in hospitalization applications.

As another potential benefit, the system and method can enable such motion quality analysis to be used in a wider variety of scenarios. A doctor is generally limited to visual inspection of motion quality while the patient is present. The system and method can enable motion quality to be tracked over longer periods of time and/or during normal life (i.e., activities outside of a doctor's office).

A biomechanical signal preferably parameterizes a biomechanical-based property of some action. More particularly, a biomechanical signal quantifies at least one aspect of motion that occurs once or repeatedly during a task. For example, in the case of walking or running, how a participant takes each step can be broken into several biomechanical signals. In a preferred implementation, the system and method preferably operate with a set of biomechanical signals, which may be customized for the particular use case. In the case where the walking gait of a patient is of interest, the biomechanical signals can include cadence, ground contact time, braking, pelvic rotation, pelvic tilt, pelvic drop, vertical oscillation of the pelvis, forward oscillation, forward velocity properties of the pelvis, step duration, stride or step length, step impact or shock, foot pronation, body loading ratio, foot lift, stride symmetry, left and right step detection, left/right foot stride asymmetry, motion paths, and/or other features. The pelvis is used as a preferred reference point. The pelvis can have a strong correlation to lower body movements and can be more isolated from upper body movements such as turning of the head and swinging of the arms. An alternative sensing reference point can be used. The sensing point is preferably centrally positioned near the median plane in the trunk portion of the body. Additional sensing points or alternative sensing points may be used depending on the activity, such as on the foot, knee or arm. The set of biomechanical signals may form a primitive set of signals from which a wide variety of activities can be monitored and analyzed. The activity is preferably characterized by predictable, repetitive, or predefined movements. A patient may be guided through some of these movements when collecting biomechanical signals. Additionally, a system of a preferred embodiment can include alternative and/or additional sensing points, which can be used for generating or refining alternative and/or additional biomechanical signals.

A biomechanical signal can be a function of time. The biomechanical signal can additionally be a real-time signal, wherein each data point is a value that corresponds to some instance of time. The real-time value can be a current value for the biomechanical property or a running average. For example real-time stride time can be averaged over a window spanning a defined set of steps (e.g., 4 steps). Averaging, smoothing, and other error correcting processes may be applied to a real-time signal.

The system and method in one preferred embodiment function to leverage the kinematic sensor data of a single sensing region. Sensing in a single region can simplify the setup of the system and the cost of the system. In one exemplary implementation, the system and method may be used within a wearable activity-tracking device. A sensor can be attached or otherwise positioned along the waist region of a participant region (e.g., the lumbar or sacral region of the back). Various use-cases could be built on top of such a system including biomechanical diagnostic tools, bio-feedback tools, gait-analysis tools, rehabilitation and physical therapy tools, coaching tools, injury prevention monitoring, performance monitoring applications, reactive sports equipment, and other suitable products. Additionally, the system may be extended to multiple point sensing. Multiple points of sensing can function to expand the number, accuracy, and variety of biomechanical signals.

As shown in FIG. 1, a system of a preferred embodiment can include an inertial measurement unit 110, a signal processor module 120, Bluetooth connectivity, and a user application. In one preferred implementation, the inertial measurement unit is integrated with an activity monitoring device 100 that includes the inertial measurement system, a housing, the signal processor module 120, memory/storage, and a communication component. The activity monitoring device 100 can additionally include any suitable components to support computational operation such as a processor, RAM, an EEPROM, user input elements (e.g., buttons, switches, capacitive sensors, touch screens, and the like), user output elements (e.g., status indicator lights, graphical display, speaker, audio jack, vibrational motor, and the like), communication components (e.g., Bluetooth LE, Zigbee, NFC, Wi-Fi, cellular data, and the like), and/or other suitable components. In an alternative embodiment the system may include an activity monitoring device 100 without a user application. The activity monitoring device 100 may directly communicate with a remote monitoring service. For example, kinematic data or biomechanical signal data could be sent over Wi-Fi or a cellular network.

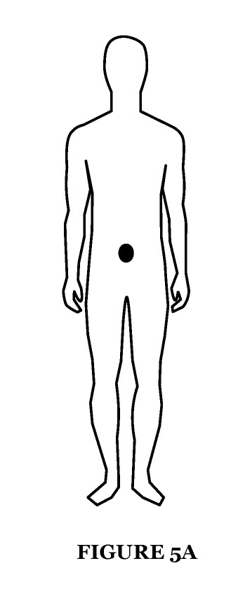

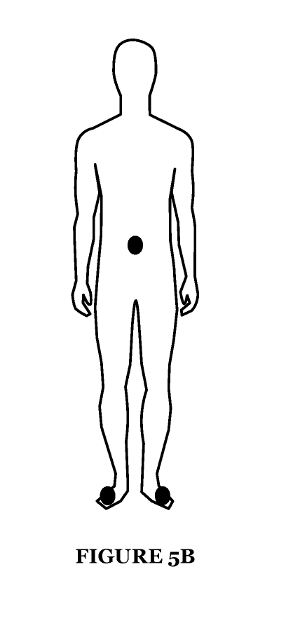

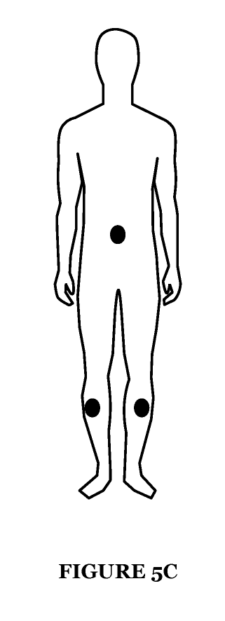

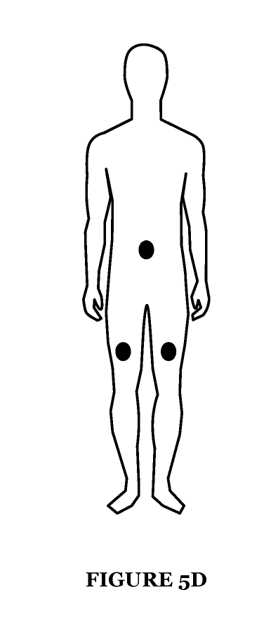

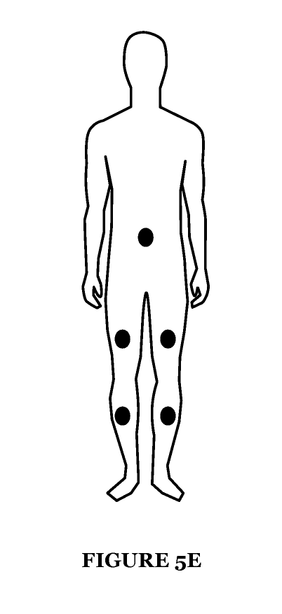

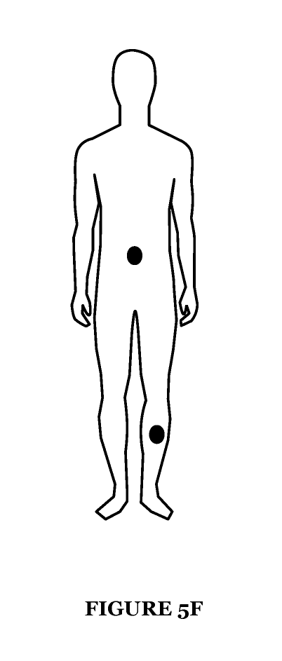

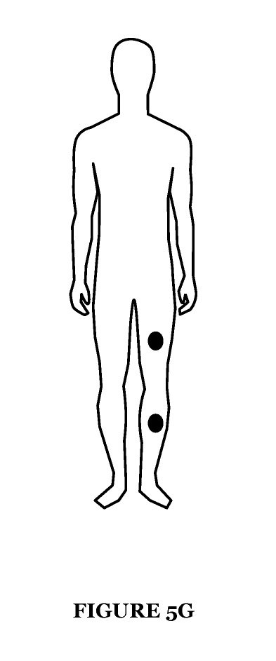

In one variation, the system includes a single activity monitor device 100. The activity monitoring system is preferably positioned in the waist region, and more specifically, the activity monitoring device 100 can be positioned along the back in the lumbar or sacral region as shown in FIG. 5A. A single activity monitoring device 100 can be used, for example, in left and right foot detection, stride analysis, braking analysis. The single point of sensing from the activity monitoring device 100 can be positioned at any suitable alternative position.

In another variation, the activity monitoring system uses a multi-point sensing approach, wherein a set of inertial measurement systems measure motion at multiple points. The points of measurement may be in the waist region, the upper leg, the lower leg, the foot, and/or any suitable location. Other points of measurement can include the upper body, the head, or portions of the arms. Various configurations of multi-point sensing can be used for sensing biomechanical signals. Different configurations may offer increased resolution, more robust sensing of one or more signals, and for detection of additional or alternative biomechanical signals. A foot activity monitoring system could be attached to or embedded in a shoe. A shank or thigh activity monitor could be strapped to the leg, embedded in an article of clothing, or positioned with any suitable approach. In one preferred variation of multi-point sensing, the system includes a pelvic activity monitoring device 100 and a right and left lower leg activity monitoring device 100. The right and left lower leg activity monitoring device 100 may be coupled to the foot as shown in FIG. 5B or the shank of each of the legs as shown in FIG. 5C. The lower leg activity monitoring device 100 can provide additional kinematic insights into how the extremities of the leg are moving. In another variation, the system can include activity monitoring devices 100 on the right and left thigh as shown in FIG. 5D. The thigh activity monitoring devices 100 can be used in combination (as shown in FIG. 5E) or as an alternative to the lower leg activity monitoring devices 100. The thigh activity monitoring devices 100 can be used to give relative rotation and motion between the thigh and the foot area and/or between the thigh and the pelvic region. While multi-point sensing preferably uses symmetrical sensing for both legs, but one alternative may use a single additional sensing point on the leg as shown in FIG. 5F. In the multiple participants embodiment, multiple activity monitor configurations may be supported. For example, a first individual may include a single activity monitor to measure walking gait biomechanical signals, and a second individual may wear a first and second activity monitor about the knee to measure knee mobility biomechanical signals as shown in FIG. 5G. The housing of the activity monitoring device 100 can enable the device to be clipped to a portion of clothing, to be worn (e.g., as a belt), or otherwise coupled to a patient. In one variation, the activity monitoring device may include an adhesive patch so that the device can be adhered directly to the body of a patient.

Multiple points of sensing can be used to obtain motion data that provides unique motion information that may be less prevalent or undetectable from just a single sensing point. Multiple points can be used in distinguishing alternative biomechanical aspects and/or to detect particular biomechanical attributes with more resolution or consistency. Multiple points may be used for detecting foot gait attributes, knee flex angle, and/or distinguishing between right and left leg actions. Single point sensing may additionally be applied to right and left leg attributes. The multiple points can be used to obtain clearer signals for particular actions such as when the foot strikes the ground. Multiple points can additionally be used in providing relative kinematics between different points of the body. The relative angular orientation and displacement can be detected between the foot, thigh, and/or pelvic region. Similarly, relative velocities between a set of activity monitoring systems can be used to generate particular biomechanical signals.

The inertial measurement unit functions to measure multiple kinematic properties of an activity. The inertial measurement unit preferably includes at least one inertial measurement unit. An inertial measurement unit can include at least one accelerometer, gyroscope, magnetometer, or other suitable inertial sensor. The inertial measurement unit preferably includes a set of sensors aligned for detection of kinematic properties along three perpendicular axes. In one variation, the inertial measurement unit is a 9-axis motion-tracking device that includes a 3-axis gyroscope, a 3-axis accelerometer, and a 3-axis magnetometer. The sensor device can additionally include an integrated processor that provides sensor fusion. Sensor fusion can combine kinematic data from the various sensors to reduce uncertainty. In this application, it may be used to estimate orientation with respect to gravity and may be used in separating forces or sensed dynamics for data from a sensor. The on-device sensor fusion may provide other suitable sensor conveniences. Alternatively, multiple distinct sensors can be combined to provide a set of kinematic measurements.

An inertial measurement unit can additionally include other sensors such as an altimeter, GPS, or any suitable sensor. Additionally the system can include a communication channel to one or more computing devices with one or more sensors. For example, an inertial measurement unit can include a Bluetooth communication channel to a smart phone, and the smart phone can track and retrieve data on geolocation, distance covered, elevation changes, and other data.

The system may additionally include and/or interface with additional physiological sensors. The additional physiological sensors could be heart rate, blood-oxygen levels, brain activity, body temperature, and other suitable biological functions.

The signal processor module 120 functions to transform sensor data generated by the inertial measurement unit no. The signal processor module 120 can include a step segmenter, a calibration module, and a set of biomechanical signal monitors. The set of biomechanical signal monitors function to output a biomechanical signal and can include variations such as a ground contact monitor, a pelvic forward velocity monitor, a pelvic vertical velocity monitor, a pelvic orientation monitor, and the like. Additional or alternative biomechanical signals may be used. In a variation using multiple sensing points, the signal processor module 120 for one of the activity monitoring devices 100 can be customized for the particular sensing point. A signal processor module 120 of a foot activity monitoring device 100 can include a step segmenter, a calibration module, a ground contact monitor, a foot pronation module, and foot velocity module. A signal processor module 120 of a thigh activity monitoring device 100 can include a step segmenter, a calibration module, a thigh orientation monitor, and a thigh velocity monitor.

The signal processor module 120 can be integrated with the activity monitoring device 100. For example, a wearable device with a battery, a communication module, and some form of user control can generate the biomechanical signals on a single device. The signal processor module 120 may alternatively be application logic operable on a secondary device such as a smart phone. In this variation, the signal processor module 120 can be integrated with the user application. In yet another variation, the signal processor module 120 can be remote processor accessible over the network. For example, biomechanical signals may be generated in the cloud, which functions to provide remote processing. Remote processing can enable large datasets to be more readily leveraged when analyzing kinematic data.

In a variation with multiple sensing points, a set of signal processor modules 120 can be included in each of the activity monitoring device 100. The generation of the biomechanical signals can be completed on each of the activity monitoring devices 100. Alternatively, kinematic data, processed data, and/or a partial set of biomechanical signal data can be communicated between the set of activity monitoring devices 100 such that data from multiple sensing points can be used in generating a biomechanical signal. One variation can include a right foot and left foot activity monitoring device 100 communicating with a parent pelvic activity monitoring device 100. The left and right activity monitor devices 100 can generate specific biomechanical signals. A subset of the biomechanical signals may be used by the parent activity monitor to generate an additional or alternative biomechanical signal. The parent pelvic activity monitoring device 100 can communicate data to a user application on a user device. Alternatively, each of the activity monitor devices 100 may directly communicate with the user application. The user application can facilitate completion or generation of all or some biomechanical signals.

The user application functions as one potential outlet of the biomechanical signal output. The user application can be any suitable type of user interface component. Preferably, the user application is a graphical user interface operable on a user computing device. The user computing device can be a smart phone, a desktop computer, a TV based computing device, a wearable computing device (e.g., a watch, glasses, etc.), or any suitable computing device. In a self-administration variation, a user application can be operable on a device of the patient. In a managed care variation, a user application can be operable on a device of a caregiver. There may be multiple versions of an application specifically designed for the particular audience. In one exemplary implementation, the user application is used to visibly represent the biomechanical signals as a graphic that characterizes the biomechanical properties of a participant's performance. In a hospitalization use case, the system can include a remote monitoring system wherein multiple patients can be monitored simultaneously. The remote monitoring system is preferably updated with motion quality and/or biomechanical signals of multiple patients. Various user applications can interface with the remote monitoring system to access the information.

The user application can alternatively be a website accessed through a client browsing device. Alternatively, the biomechanical signals may be accessed synchronously or asynchronously through an application programming interface (API). In one variation, the user application can be integrated with the inertial measurement unit 110 or part of a secondary device. For example, the device of the inertial measurement unit 110 may include a set of audio or visual outputs that can be used in representing the biomechanical signals. Haptic feedback elements and other forms of user feedback may additionally be controlled by the user application.

### 2. Method

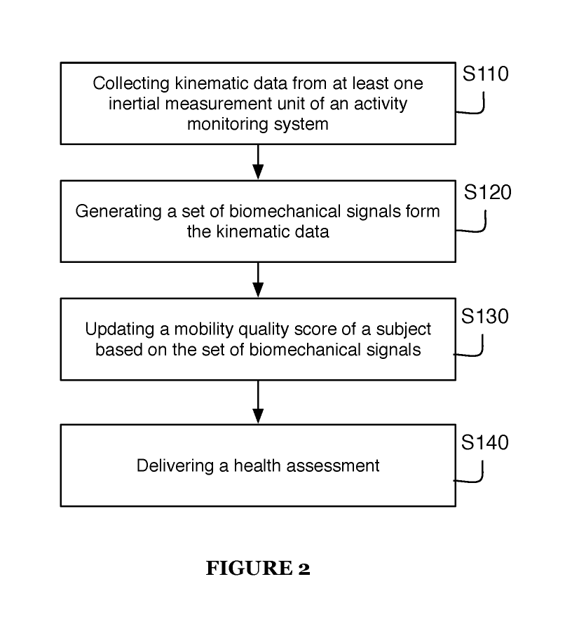

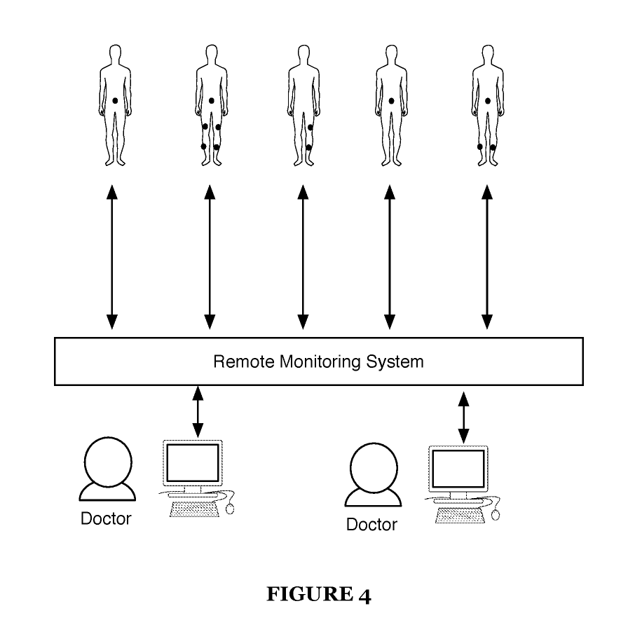

As shown in FIG. 2, a method for applying biomechanical characterizations to patient care of a preferred embodiment can include collecting kinematic data from at least one inertial measurement unit of an activity monitoring system S110; generating a set of biomechanical signals form the kinematic data S120; updating a mobility quality score of a subject based on the set of biomechanical signals S130; and delivering a health assessment S140. Preferably, the method is applied in connection to at least one treatment stage. In one variation, the method can be applied in translating kinematic data into a tangible health-related directive. A health-related directive may come in the form of a health report that communicates information to a physician to aid in guiding patient care decisions. Health-related directives may alternatively come in other forms such as digital instructions to one or more systems or devices. In another variation, the method can include one or more iterations at various treatment stages for progressive or comparative analysis. As described above, the method can use kinematic sensor data in generating detailed biomechanical characterizations of body motion. The method is preferably used in a gait sensing mode wherein how a patient walks, “shuffles”, runs, or otherwise moves on his feet can be monitored from a biomechanical perspective. The method can additionally include other sensing modes such as a posture mode, repose mode (e.g., used during bed rest or at night), and/or a particular exercise mode. The biomechanical characterizations of body motion may be applied to the medical space in improving the care of one or more patients. The method may be used in a variety of use cases that can include: a tool in augmenting medical treatment (e.g., drug treatment, physical therapy, medical device usage, and the like), a treatment analysis tool, a diagnostics tool, event detection, a hospitalization tool, and/or any suitable use case. In one variation, the method may be used with a single patient. In another variation, the method may be used across a set of different patients to more broadly help patients in a hospitalization or group scenario as shown in FIG. 4.

The method is preferably implemented by a system as described above. Preferably, the method is implemented in association with at least one positioned inertial measurement unit and more preferably at least one point inertial measurement unit of an activity monitoring device that is substantially physically coupled to the waist region of a participant. For example, a sensing device may be attached to the back portion of a waistband of a garment. The location of the activity monitoring device could alternatively be at various locations such as on the chest, the head or neck, a leg or arm, or any suitable location. Additionally, the method can involve sensing kinematic data from multiple points, and applying that kinematic data to generating the set of biomechanical signals.

In one variation, the method is implemented at least in part by a computing device that includes or is connected to a sensing system. In another variation, the method is implemented on a native application operable on a personal computing device (e.g., smart phone, wearable computing device, or personal computer). In yet another variation, the method can be implemented in the cloud on a remote server. The method may alternatively be implemented through any suitable system.

Additionally, the method can be used in combination with a medical device. In one example, the method can be used in combination with a pain medication treatment device to regulate or signal dosage amounts or limits. Mobility quality can be used as a metric by which medication can be adjusted. The method could generate dosage recommendations and adjustments as a form of mobility quality directed medication titration. In the application of pain medicine, mobility quality can be related to pain and thus mobility quality can be used to regulate or guide pain medicine requirements and/or limits. This could be used in determining preferred medication limits but could also be used in gradually limiting medication. In another example, the method can be used in combination with a remote patient monitoring system, which may be used in a hospital in monitoring care for multiple patients. Nurses, doctors, and/or other medical workers could be directed to different patients according to analysis of mobility quality and/or biomechanical signals.

Block S110, which includes collecting kinematic data from at least one inertial measurement unit of an activity monitoring system, functions to sense, detect, or otherwise obtain sensor data relating to motion of a patient. In one variation, data of the kinematic data streams is raw, unprocessed sensor data as detected from a sensor device. Raw sensor data can be collected directly from the sensing device, but the raw sensor data may alternatively be collected from an intermediary data source. In another variation, the data can be pre-processed. For example, data can be filtered, error corrected, or otherwise transformed. In one variation, in-hardware sensor fusion is performed by an on-device processor of the inertial measurement unit. The kinematic data is preferably calibrated to some reference orientation. In one variation, automatic calibration may be used as described in U.S. patent application Ser. No. 15/454,514 filed on 9 Mar. 2017, which is hereby incorporated in its entirety by this reference.

Any suitable pre-processing may additionally be applied to the data during the method.

The individual kinematic data streams preferably correspond to distinct kinematic measurements along a defined axis. The kinematic measurements are preferably along a set of orthonormal axes (e.g., an x, y, z coordinate plane). As described below, the axis of measurements may not be physically restrained to be aligned with a preferred or assumed coordinate system of the activity. Accordingly, the axis of measurement by one or more sensor(s) may be calibrated for analysis by calibrating the orientation of the kinematic data stream. One, two, or all three axes may share some or all features of the calibration, or be calibrated independently. The kinematic measurements can include acceleration, velocity, displacement, force, rotational acceleration, rotational displacement, tilt/angle, and/or any suitable metric corresponding to a kinematic property of an activity. Preferably, a sensing device provides acceleration as detected by an accelerometer and angular velocity as detected by a gyroscope along three orthonormal axes. The set of kinematic data streams preferably includes acceleration in any orthonormal set of axes in three-dimensional space, herein denoted as x, y, z axes, and angular velocity about the x, y, and z axes. Additionally, the sensing device may detect magnetic field through a three-axis magnetometer. Collecting kinematic data can additionally include normalizing the kinematic data to a standardized format, which can be used in generating the biomechanical signals of Block S120.

Normalizing the set of kinematic data streams functions to set a reference context in which the kinematic data is collected and analyzed. Normalizing can involve standardizing the kinematic data and calibrating the kinematic data to a reference orientation such as a coordinate system of the participant. The nature of normalization can be customized depending on the sensing mode. For example, in a walking mode, normalizing the set of kinematic data streams can include adapting orientation of kinematic data sensing to a participant orientation, determining a base pelvic tilt position, and segmenting at least a subset of the kinematic data streams by detected steps. The inertial measurement unit is preferably part of a handheld device that can be attached or otherwise fixed into a certain position during an activity. That position can be static during the activity but may also be perturbed and change. Preferably, the inertial measurement unit is positioned in the waist region and more specifically in the lumbar or sacral region of the back. Additional inertial measurement units can be positioned at varying points to provide kinematic data streams for other portions of the body. The foot, the shank of the leg, and the thigh are three optional points. Inertial measurement units can capture kinematic data streams for each of a right leg and a left leg.

The method can additionally include measuring at least a second physiological signal to supplement the biomechanical signals. The physiological signals can include a heart rate, EMG signal, breathing rate, perspiration, blood chemistry reading, and/or any suitable physiological measurement. A physiological signal can be used in combination with the biomechanical logic system when applying the method to a particular use case.

Block S120, which includes generating a set of biomechanical signal from the kinematic data, functions to characterize at least one aspect of how a patient is moving. One or more different biomechanical signals may be produced depending on the use case of the method and condition of the patient. The biomechanical signals may characterize the motion patterns of a particular body part. For example, the motion of a foot, a knee, pelvis or any suitable body part can be monitored and characterized in one or more biomechanical signals. The biomechanical signals may alternatively characterize the biomechanical relationship of multiple body parts. For example, the biomechanical signals can show imbalances in a patient's gate or a comparison of mobility between two joints. The biomechanical signals may alternatively characterize the mobility quality of the particular body part, joint, or overall walking gait over time.

The set of biomechanical signals preferably functions to parameterize a set of primitives from which the motion properties of an activity can be monitored and acted on. The biomechanical signals are preferably specific for particular activities and/or applications. As will be described below, the method may enable dynamic selection of different operating modes so that an activity monitoring device can adapt to different activities or be used in different healthcare applications. The set of biomechanical signals may alternatively be preconfigured.

In a walking sensing mode the biomechanical signals can be based on step-wise windows of the kinematic data streams—looking at single steps, consecutive steps, or a sequence of steps. In one variation, generating a set of biomechanical signals can include generating a set of stride-based biomechanical signals comprising segmenting kinematic data by steps and for at least a subset of the stride-based biomechanical signals generating a biomechanical signal based on step biomechanical properties. Segmenting can be performed for walking and/or running. In one variation steps can be segmented and counted according to threshold or zero crossings of vertical velocity. A preferred approach, however, includes counting vertical velocity extrema. Another preferred approach includes counting extrema exceeding a minimum amplitude requirement in the filtered, three-dimensional acceleration magnitude as measured by the sensor. The set of stride-based biomechanical signals can include cadence, ground contact time, braking, pelvic rotation, pelvic tilt, pelvic drop, vertical oscillation of the pelvis, lateral oscillation of the pelvis, forward oscillation, upper body trunk lean, forward velocity properties of the pelvis, step duration, stride or step length, step impact or shock, foot pronation, body loading ratio, foot lift, step and/or stride length, swing time, double-stance time, leg lift response time, activity transition time, stride symmetry, left and right step detection, motion paths, and/or other features. Other health related biomechanical signals can relate to balance, turn speed, tremor quantification, shuffle detection, variability or consistency of a biomechanical property, and/or other suitable health related biomechanical properties. In one variation, the biomechanical signals may be generated in a substantially similar manner to those discussed in U.S. patent application Ser. No. 15/282,998 filed on 30 Sep. 2016, which is hereby incorporated in its entirety.

Cadence can be characterized as the step rate of the participant.

Ground contact time is a measure of how long a foot is in contact with the ground during a step. The ground contact time can be a time duration, a percent or ratio of ground contact compared to the step duration, a comparison of right and left ground contact time or any suitable characterization.

Braking or the intra-step change in forward velocity is the change in the deceleration in the direction of motion that occurs on ground contact. In one variation, Braking is characterized as the difference between the minimum velocity and maximum velocity within a step, or the difference between the minimum velocity and the average velocity within a step. Braking can alternatively be characterized as the difference between the minimal velocity point and the average difference between the maximum and minimum velocity. A step impact signal may be a characterization of the timing and/or properties relating to the dynamics of a foot contacting the ground.

Pelvic dynamics can be represented in several different biomechanical signals including pelvic rotation, pelvic tilt, and pelvic drop. Pelvic rotation (i.e., yaw) can characterize the rotation in the transverse plane (i.e., rotation about a vertical axis). Pelvic tilt (i.e., pitch) can be characterized as rotation in the sagittal plane (i.e., rotation about a lateral axis). Pelvic drop (i.e., roll) can be characterized as rotation in the coronal plane (i.e., rotation about the forward-backward axis).

Upper body trunk lean is a characterization of the amount a user leans forward, backward, left or right when walking.

Vertical oscillation of the pelvis is characterization of the up and down bounce during a step (e.g., the bounce of a step).

Lateral oscillation of the pelvis is the characterization of the side-to-side displacement during a stride.

Forward velocity properties of the pelvis or the forward oscillation can be one or more signals characterizing the oscillation of distance over a step or stride, velocity, maximum velocity, minimum velocity, average velocity, or any suitable property of forward kinematic properties of the pelvis.

Step duration could be the amount of time to take one step. Stride duration could similarly be used, wherein a stride includes two consecutive steps.

Foot pronation could be a characterization of the angle of a foot during a stride or at some point of a stride. Similarly foot contact angle can be the amount of rotation in the foot on ground contact. Foot impact is the upward deceleration that is experienced occurring during ground contact. The body-loading ratio can be used in classifying heel, midfoot, and forefoot strikers. The foot lift can be the vertical displacement of each foot. The motion path can be a position over time map for at least one point of the runner's body. The position is preferably measured relative to the athlete. The position can be measured in one, two, or three dimensions. As a feature, the motion path can be characterized by different parameters such as consistency, range of motion in various directions, and other suitable properties. In another variation, a motion path can be compared based on its shape.

The foot lift can be the vertical displacement of each foot.

Step length is the forward displacement of each foot. Stride length is the forward displacement of two consecutive steps of the right and left foot.

Swing time is the amount of time each foot is in the air. Ground contact time is the amount of time the foot is in contact with the ground.

Double-stance time is the amount of time both feet are simultaneously on the ground during a walking gait cycle.

Leg lift response time is the amount of time it takes for a patient to lift their leg when prompted.

Activity transition time preferably characterizes the time between different activities such as lying down, sitting, standing, walking, and the like. Sit-to-stand transition in particular may be useful in the health space. A sit-to-stand transition is the amount of time it takes to transition from a sitting state to a standing state.

Stride symmetry can be a measure of imbalances between different steps. It can account for various factors such as stride length, step duration, pelvic rotation, and/or other factors. In one implementation, it can be characterized as a ratio or side bias where zero may represent balanced symmetry and a negative value or a positive value may represent left and right biases respectively. Symmetry could additionally be measured for different activities such as posture symmetry (degree of leaning to one or another side) when standing.

Left and right step detection can function to detect individual steps. Any of the biomechanical signals could additionally be characterized for left and right sides.

The motion path can be a position over time map for at least one point. Participants will generally have movement patterns that are unique and generally consistent between activities with similar conditions.

Balance can be a measure of posture or motion stability when walking, running, or standing. Pelvic coronal drop, pelvic transverse rotation and pelvis lateral oscillation values can help measure balance.

Turn speed can characterize properties relating to turns by a patient. In one variation, turn speed can be the amount of time to turn. Additionally or alternatively turn speed can be characterized by peak velocity of turn, and/or average velocity of turn when a user makes a turn in their gait cycle.

Tremor quantification can include detecting tremors but can additionally be used in measuring duration, frequency response components, and magnitude of tremors. In some cases, a tremor activity by a patient may have a frequency response such as between 4 Hz and 10 Hz, which could be characterized by the frequency response components. Additionally, range of motion may also quantify the tremor magnitude.

Shuffle detection can be a characterization of shuffling gate when moving. Shuffling may be a walking motion that lacks the vertical displacement of the feet when walking.

Biomechanics variability or consistency can characterize variability or consistency of a biomechanical property such as of the biomechanical signals discussed herein. Cadence variability may be one exemplary type of biomechanical variability signal, but any suitable biomechanical property could be analyzed from a variability perspective. For example, high variance in walking biomechanical signals such as cadence, speed, and pelvis rotation values has been correlated with poor mobility quality.

The above biomechanical signals can have particular applicability to walking, running, and standing use-cases. Alternative use cases may use alternative biomechanical signals relating to acceleration, deceleration, change of direction, jump duration, and other suitable properties of performing some activity.

In one particular application, producing biomechanical signals is customized for monitoring imbalances in a patient's gate (e.g., a limp). The set of biomechanical signals preferably include signals for the right leg and the left leg. The biomechanical signals for the right and left can be compared to ascertain the nature of an imbalance. For example, the ground contact time for the right leg can be compared to the left leg. The magnitude of the difference in the ground contact time can correspond to the severity of a limp. In addition, the location of the injury can be detected depending on the gait signature. Furthermore, recovery progress can be objectively observed overtime and shared with the physician or nurse.

In another application, producing biomechanical signals is customized for monitoring mobility of a targeted joint such as a hip, knee, or ankle. A multi-point sensing approach may be used to target a particular set of biomechanical signals wherein kinematic data is collected from at least two inertial measurement units. In this variation, generating a set of biomechanical signals can include generating a relative measurement of motion between a first inertial measurement unit and a second inertial measurement unit. In one variation, this relative measurement can be a relative displacement observed between the two inertial measurement units. In another variation, the relative measurement can be an angular displacement. This may be able to create a biomechanical signal that reflects a range of motion for one or more body parts. For a joint, a first activity monitor can be at a point on one segment of the joint and a second activity monitor can be at a point on a second segment of the joint. The relative kinematic data from the first and second activity monitor can be used in understanding the biomechanics of the joint such as the range or rotation, the speed of rotation, the resting angle, and/or other suitable properties.

In one implementation, a physical therapy rehabilitation program can be embedded into a software application that guides the patient to do in-home exercises for the knee. The program guides the user to place a knee sleeve with sensors on the top and bottom of the knee joint. The user slowly rotates their knee up and down, left and right to the point before pain sets in. The rehabilitation application records the range of motion angles and suggests specific stretches. The information can be sent to a remote monitoring backend where the physical therapists have the ability to view data and update flexibility and strengthening plans.

Block S130, which includes updating a mobility quality score of a subject based on the set of biomechanical signals, functions to analyze the set of biomechanical signals to derive some assessment of the health or value of how the patient is moving. In one variation, the mobility quality score can be an abstraction of the set of biomechanical signals. Analysis of the biomechanical signals can include analysis of current or recent biomechanical signals but can additionally be a historical analysis. For example, trends or patterns of biomechanical signals may be used in part in characterizing the biomechanics of the patient and in updating the mobility quality score. In other variations the mobility quality score can be a modeled representation of multiple properties. The various properties included in a mobility quality score can include in part a subset of the biomechanical signals, a subset of processed versions of biomechanical signals, and/or any suitable property. As discussed, alternative biological signals such as heart rate, respiratory rate, blood chemistry and/or other properties may be collected. These biological signals can be a factor when updating a mobility quality score. While the set of biomechanical signals may comprise time-series data, that mobility quality score can look at the biomechanical signal values across time.

An algorithm or other suitable process can be used in translating and/or characterizing the metrics from the set of biomechanical signals to a mobility quality score. Updating the mobility quality score may additionally or alternatively include updating analysis of the mobility quality. Updating analysis preferably includes classifying mobility of a patient. Updating analysis of the mobility quality can generally involve comparing the biomechanical signals and/or analysis of the biomechanical signals. The recorded biomechanical signals could be compared to previous samples of biomechanical signals. For example, during a rehabilitation use case, the time ordered set of biomechanical signal samples could be analyzed for a trend of improvement. The recorded biomechanical signals may alternatively be compared or analyzed based on expected values.

In one implementation, a generalized mobility quality score can be based on a number of biomechanical signals averaged together and compared to a larger population-based reference of similar biomechanical signals from a healthy and relevant demographic. For example, the patient's mobility quality can be compared to the biomechanical averages of biomechanical signals that characterize overall walking gait consistency, balance, speed, and dexterity. These averaged values can be averaged over hour, days, weeks, or any suitable time window and compared to an averaged value based on healthy individuals that are of similar weight, height, gender, age or any other relevant demographic. This allows the patient to understand how his mobility quality compares to a relevant healthy population and empowers the patient to measure and understand how changes to habits can improve (or degrade) personal mobility quality.

Additionally or alternatively, the mobility quality score may be organized into specific categories such as walking consistency, speed, and balance. A walking consistency quality score may include measuring the overall consistency of all biomechanical signals of a patient, or averaged consistency across a group of biomechanical signals such as a patient's cadence, vertical oscillation of the pelvis, and rotations of the pelvis across each step or stride or set of strides. Alternative consistency quality scores may also focus on a specific biomechanical signal over time, especially if a physician has isolated a specific metric to monitor for rehabilitation. Another mobility quality category can be focused on the average speed of a patient over 1 step, 1 stride, 5 strides or any number of averaged steps or strides. The average speed of a user is another good indicator for measuring mobility quality as patients generally regain the ability to walk faster as their rehabilitation progresses. A balance quality score can be another mobility quality category as patients with good balance are less likely to fall compared to patients with poor balance. For example, a balance quality score may be calculated with averages of pelvic coronal drop, pelvic rotation and pelvic lateral oscillation, which are biomechanical signals that help characterize the balance and stability of an individual when walking. Additional mobility quality score categories can also be based on any other single biomechanical signal or group of signals.

In one variation, heuristics may be used in checking various biomechanical signal thresholds and/or conditions. In another variation, algorithmic analysis or machine learning can be applied. For example, a classifier can be set to automatically classify the set of biomechanical signals into five mobility ratings ranging from good to bad. This can be treated as a supervised classification problem which may utilize neural networks, radial basis functions, support vector machines, k-nearest neighbors, and the like. Any suitable approach may be used in any suitable combination.

Block S140, which includes delivering a health assessment, functions to use the biomechanical signals and the resulting mobility quality score to drive various health related events. Delivering a health assessment is an application that can be used in updating a user feedback device, medical device, or other output. Delivering a health assessment can include generating a report, transmitting an electronic communication, directing an action in one or more devices, or applying the results in any suitable manner.

Generating a report can involve translating the mobility quality score into a graphical representation. A qualitative assessment based on the biomechanical signals can similarly be generated and used in the report. A report could be used by a doctor, a patient, or an electronic device. In one variation, the health assessment is a recovery report. In this variation, collection of the kinematic data, generation of the biomechanical signals and the updating of a mobility quality score are performed during the recovery stage of a treatment. The recovery report can include recovery rate, a recovery comparison, and/or an expected recovery time for a particular treatment. A recovery comparison can be a comparison to the patient's original mobility quality and biomechanics. A recovery comparison may alternatively be a comparison to other patients. Additionally or alternatively a recovery comparison can reference a target mobility quality score and/or biomechanical signal values.

Transmitting an electronic communication can be used in remotely monitoring a patient. In one example, transmitting an electronic communication can be used in a hospitalization use case. Similarly directing an action can be an electronic communication used in altering the operating mode of a device or system. In one example, the action may be altering delivery of treatment by a medical device. In another example, the action may be triggering an alarm in a fall prevention system.

In one variation, block S140 preferably includes executing a biomechanical logic system. A biomechanical logic system can analyze and execute any suitable business logic driven by the mobility quality score. A biomechanical logic system can be used to provide on-demand feedback to a user, generate exercise/tasks, generate activity performance reports, or perform any suitable task. In one variation, the biomechanical logic system can operate on a personal computing device of the patient. Alternatively, the biomechanical logic system can be accessible by an administrator such as a doctor or personal trainer.

Execution of the method and in particular delivering of a health assessment may be customized depending on the particular use case. In particular the method can be used during treatment administration, diagnostics, event detection, and/or hospitalization.

### Treatment Administration Use Case

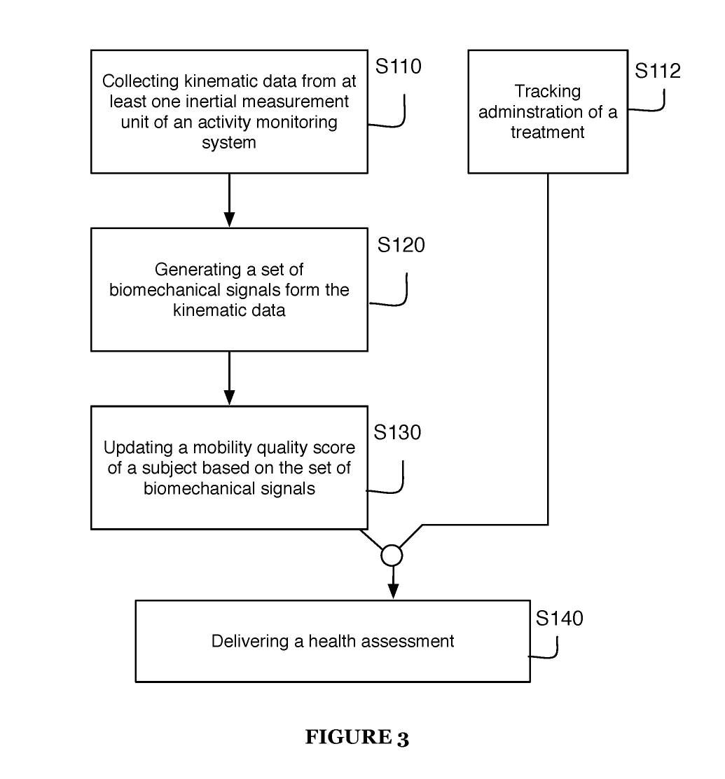

When applied to treatment administration, the method may additionally include tracking administration of a treatment S112 as shown in FIG. 3. Preferably tracking administration of a treatment can include tracking timing of treatment and properties of the treatment. Properties of a drug treatment may include the drug and dosage. Properties of a medical device treatment may include the settings and duration of medical device usage. Properties of physical therapy may include the exercises performed, the number of repetitions, and the like. The administration of the treatment is preferably performed over a sustained period. Kinematic data is preferably collected at multiple times either continuously or in distinct time windows over the course of the treatment, and biomechanical signals are similarly generated from the kinematic data collected at different times. The delivered health assessment can be an analysis of the treatment based in part on the motion quality in comparison to the administration of the treatment. In particular, changes in mobility quality can be monitored. The delivered health assessment can additionally include a report on treatment recommendations. The treatment recommendation can be a recommended or prescribed quantity of the treatment. Such recommendations can be used to generate or approve individual prescriptions. Such medication adjustments can enable treatments to be adjusted specifically to a patient based in part on the patient's mobility. For example, a drug treatment recommendation may specify the dosage amount for a prescription. In another example, a physical therapy treatment recommendation may specify particular physical therapy exercises. Similarly, the treatment recommendation may specify timing of a treatment. The method may be used in various forms of treatment, drug administration, physical therapy, and medical device treatment

In the drug administration variation, the biomechanical signals of an individual can be monitored before and after administration of a drug. The biomechanical impact of the drug can be monitored and used in determining the effectiveness of the drug and/or altering the medication treatment. The biomechanical impact of the drug can alternatively be monitored and used to determine the amount and/or timing of a subsequent dose. As one potential benefit, use in drug administration, the method can significantly bring down the time to stabilize medical titration. In some cases, patients do not get the right prescriptions for months of trial and error. This timeframe can become significantly reduced with monitored health assessments. For example, effects of a muscle relaxing or painkiller pharmaceutical could be evident in the movements of a patient. As the drug wears off, the movements of the patient may gradually transition back to an unmediated state. The system and method could be used in targeting drug treatments based on the biomechanical impact. Additionally, the method could be applied to measure drug effectiveness in reducing recovery time.

Similarly, the use of a medical device could be guided based on the system and method. A medical device with periodic usage such as a brace or Transcutaneous Electrical Nerve Stimulation (TENS) device may particularly benefit from such an application. The system and method can be used with the medical device in a manner substantially similar to the administration of a drug. The long term and short-term effects of a medical device can be monitored and used in determining how a medical device is used. The system and method can be used in recommending timing, duration, and nature of usage for the medical device. In another variation, exercises, diets, and/or other activities may be recommended based on the system and method. In one variation, the treatment recommendation is directly transmitted to the medical device to update the operating state of that medical device. For example, a TENS unit may be at least partially controlled based on the health assessment of the method.

In another use case, the system and method can be applied to rehabilitation treatment of a patient. Rehabilitation could be from some post-surgery operation, post injury, or when recovering from any suitable condition. The rehabilitation is preferably for some injury or symptom that is affecting the mobility of the patient. The biomechanical signals can be used during rehabilitation to address kinematic imbalances, range of motion, speed of motion, strength of motion, and/or other properties of how the patient is moving.

In the rehabilitation use case, a patient can wear and use the system multiple times over a recovery period. The administration of physical therapy treatment can be tracked and compared to changes in mobility quality. In one variation, the patient periodically records activity to provide a sample of the biomechanical characteristics at that point in time. The patient could record the biomechanical signals with the help of a doctor or trainer. For example, a trainer could guide the patient to perform particular exercises and record the biomechanical signals as that exercise is performed. These recorded biomechanical signals can then be used as a target state for the patient as the patient continues to perform exercises on their own, for example, in their home environment. This can enable the patient to follow a path that the physical therapist intended. The therapist can monitor progress remotely and adjust the training plan remotely based on patient progress. In another variation the patient wears the system continuously. A set of biomechanical signal samples from different points in time can be used in understanding the rehabilitation process. As an example, depending on the biomechanical signals and/or mobility quality detected at various points in time (e.g. signifying changes in range of motion, ambulatory state, or sedentary activity), this may indicate that the rehabilitation process is progressing slower or faster than anticipated. In one variation, the health assessment can be a rehabilitation analysis. The rehabilitation analysis could include a recovery progress report, recovery time estimation, physical recovery percentage, incremental improvement reports, and/or any suitable information. The analysis and recommendations may also be generated using machine learning methods that are based on a growing dataset of patient recovery progress and history. The analysis may help guide the decision-making processes of physicians for their patients. For example, based on a generated report, the patient may have worsening ambulation and range of motion, prompting the physician to bring the patient in earlier for a visit. The method may include triggering an alert when such health concerns are identified so that a physician or patient can take appropriate action. In one implementation, the biomechanical signals can be compared to target biomechanical signals. The target biomechanical signals could be theoretical, measured, or generated. The biomechanical signals measured by the patient from before the condition could be used as a target if such biomechanical signals are available. Additionally, current and previous continuous activity measurements or biomechanical signals can be compared to denote a change and either improvement or worsening of symptoms. Alternatively, biomechanical signals based on others with a similar health profile may be used. All of these implementations enable benchmarking of the patient's rehabilitation progress to a reference standard whether that is himself or a group of patients similar to himself. This could automatically inform the patient, family members and physician about patient progress. In one variation, milestones could be generated based on expected rehabilitation progress. The milestones can act as incremental goals. These milestones may be generated from anonymized biomechanical data from previous patients undergoing similar rehabilitation progress or protocols. The health assessment may additionally include treatment recommendations such as the timing of the physical therapy sessions and which exercises or treatments are working. The health assessment can additionally be used in monitoring patient compliance for performing different exercises. The health assessment may also include both the quality and quantity of exercises performed.

The system and method can additionally be used for real-time rehabilitation. In the example with the trainer, the trainer could coach an individual on performing a task and use the real-time biomechanical feedback to coach the patient. In a training session, a patient could use the system and method to see real tangible improvements. In addition, a physical therapy software application or virtual coach can provide the patient with real-time guidance and exercises. The application can reward the patient for achieving various exercises or range of motion objectives. The application could be turned into a game with the user performing exercises to complete virtual objectives, making exercises fun and engaging to help counteract traditionally poor patient compliance.

The system and method can enable a data driven approach to rehabilitation. Data from one or more patients can be applied to determine recommended approaches and to customize rehabilitation steps for an individual. Patients may have unique individual responses to different exercises and rehabilitation steps. The system and method can provide a concrete metric to judge the effectiveness of techniques and adjust rehabilitation to achieve improved results. For example, the effectiveness of surgeries for specific issues and the effectiveness of particular doctors performing said surgeries can be analyzed through the method pre and post-surgery.

Treatment administration can additionally relate to surgery analysis tools. The method can be used during pre-surgery in assessing the need for a treatment, possibly compared to other treatments. This may be determined based on specific movement patterns resulting in an understanding of whether specific surgeries (or other treatments) would be most beneficial for the patient. The method can alternatively be used in post-surgery stage in monitoring recovery progress. Range of motion and ambulation can be very different for patients who are recovering, depending on a variety of factors, including the success of the surgery, the ability to tolerate pain, and the patient's reluctance to move post surgery. The method may also be used during a pre-surgery treatment stage and a post-surgery treatment stage, wherein a mobility quality comparison can be applied in assessing recovery.

In the pre-surgery use case, the health assessment preferably includes a metric for predicted treatment impact. In some implementations, the predicted treatment impact may be represented by a surgery recommendation score. A report on the predicted treatment impact could similarly be used during the assessment stage for any treatment such as when considering drug treatments, physical therapy, or other suitable forms of treatments. In one implementation, the health assessment could predict changes in mobility, and could state the probability of improving mobility. For example, a report may indicate that surgery could increase range of motion by 10 degrees. Predictions may be generated based on data collected from patients where the method is used during pre- and post-surgery treatment. The predictions could be based on the mobility changes observed by patients of a similar biomechanical signal profile during a pre-surgery stage and post-surgery stage and/or changes observed by patients of a similar demographic or profile.

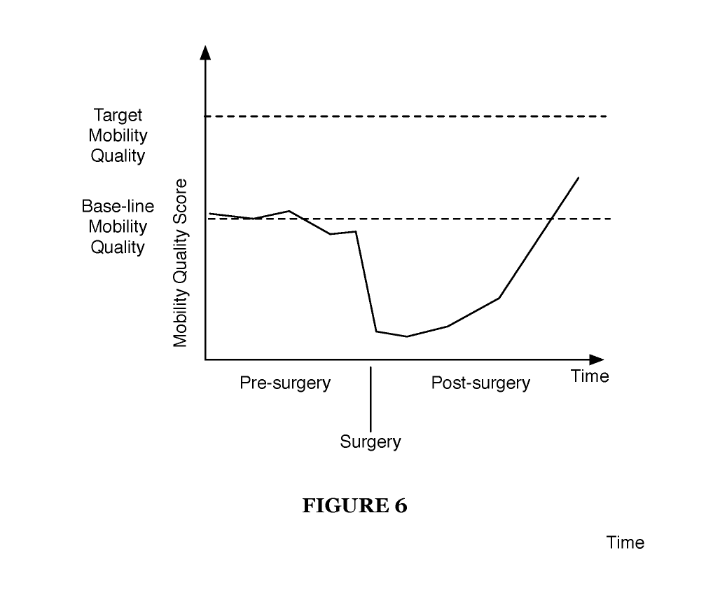

In the post-surgery use case, the efficacy of the surgery can be measured by monitoring the changes in movement patterns as reflected in the mobility quality score and the biomechanical signals. When mobility quality data is available from a pre-surgery treatment stage, block S140 can include generating a report on mobility recovery with a comparison to prior mobility quality as shown in FIG. 6. More specifically, delivering the health assessment can include comparing current motion quality score from a current post-surgery stage to a motion quality score from a pre-surgery stage as an assessment of mobility recovery. The pre-surgery mobility quality score preferably provides a base-line for a comparison.

### Diagnostics Use Case

In another use case, the system and method can be used as a tool in guiding diagnosis of medical issues. As one potential benefit, the system and method can offer a non-invasive technique to identifying underlying physiological and/or biomechanical problems with an individual, which may be used in diagnosing movement disorders such as Parkinson's disease. In some cases, a doctor can detect the onset of a medical issue through visual inspection such as by watching a patient walk in a straight line and noticing biomechanical tendencies. However, a visual inspection is prone to numerous errors and is not sensitive to subtle signs. The system and method may enable earlier detection and more reliable detection of such problems. Secondly, the system and method may enable more objective and consistent results across care providers. As a third potential benefit, the system and method can be used remotely without needing the patient to be in the presence of a doctor. A patient equipped with the system can perform the necessary activities to collect the data. In this variation, the health assessment can be a diagnosis report. In one implementation, the diagnosis report can score the patient for one or more health issues. The collected information could then be transferred to a doctor or caretaker for further analysis. The patient could automatically be alerted to health warnings without interacting with a doctor.

As a diagnosis tool, a participant can wear an activity monitor and then collect a set of biomechanical signal samples while performing one or more activities. In one variation, a participant can be guided through a set of different activities. In performing the method for a diagnosis treatment stage, the method can include directing actions by a patient and generating biomechanical signals from kinematic data during the directed actions. Directing actions can include displaying or otherwise presenting instructions for a user or a medical worker administering the test. The actions can include physical motions such as walking 10 steps, standing up, sitting down, bending over, and the like. The set of biomechanical signals collected and/or the nature of the analysis of the biomechanical signals can be customized for each activity. For example, a user could be guided to walk in a straight line, to walk backwards in a straight line, to touch his toes, to transition between a sitting and a standing position, and/or perform any suitable activity. In another variation, a participant may use the system for an extended period of time so as to provide a better reflection of normal activity. The nature of the system and method can enable a patient to take the system home for a period of time to collect a sufficient sample of biomechanical signals. Home usage can additionally be used during routine checkups such that normal activity can be determined. Once a normal set of biomechanical signals have been collected, the method could more easily detect abnormal activity. In one variation, the diagnosis could be a real-time diagnosis such that an alert could be triggered at the moment an event occurs. For example, the normal activity of an individual could be established and then any abrupt falls could be automatically detected and reported.

In the method, a set of biomechanical signal samplings is collected for each activity and then processing the biomechanical signal samples. In one variation, processing can include comparing to a set of expected values. The expected values could be measured or calculated for a user of similar health status (e.g., age, weight, height, medical conditions, etc.) or compared to relevant reference of healthy individuals in the same demographic. The method can include processing the biomechanical signals for particular patterns. The patterns can correspond to particular warnings or diagnosis. Alternatively, machine learning or other forms of machine intelligence can be used in identifying patterns, classifying data, or performing other aspects of analysis.

In one variation, use of the system and method as a diagnosis tool can be used as part of a pre-surgery process as discussed above. A patient considering surgery may be monitored by the device, and then the biomechanical properties of the injury necessitating medical intervention can be characterized. A patient and/or doctor can then use this information to consider the medical options for treatment. The method could generate a prediction of recovery expectations including amount of recovery and rate of recovery. Additionally, the system and method may be used in recommending a particular type of treatment, which can include physical therapy, surgery, medical device, medication, or other suitable recommendations.

### Event Detection Use Cases

In another use case, the method can be used for event detection. During event detection, the patient is preferably continuously monitored. When used in event detection, updating the mobility quality score can include detecting an event risk scenario through the biomechanical signals. An event risk scenario may be indicated by detecting changes in mobility quality score and/or detecting patterns in the biomechanical signals that are associated with a risk of the event. In one variation, heuristics may be used in checking various biomechanical signal thresholds and/or conditions for signs of particular events. In one implementation, a heuristic based mobility quality score for quantifying fall risk can be calculated by increased prevalence of specific mobility quality indicators like shuffle gait detection, low vertical displacement of the foot, and high variances in walking gait balance. In another variation, algorithmic analysis or machine learning can be applied. For example, a classifier can be set to automatically identify various event types. This can be treated as a supervised classification problem which may utilize neural networks, radial basis functions, support vector machines, k-nearest neighbors, and the like. Any suitable approach may be used in any suitable combination. In one variation, the method may include receiving an event report that is logged by a health care representative. The event report preferably includes the event type and the time of the event. This can be used in training the system wherein future events may be identified by looking for biomechanical signal patterns that were present leading up to previous events. When risk of an event is detected, delivering the health assessment can include triggering an alert. The alert may be used in communicating to health care worker to come assist the patient. The alert could alternatively be feedback delivered to a patient so they can be made more aware of the risk. Event detection can be used in predicting fall risk and detecting falls. By triggering alerts, falls can be prevented prior to occurring.

### Hospitalization and Quality of Care Use Cases

The system and method can alternatively be applied to hospitalization monitoring, which functions to act as a tool for monitoring a plurality of individuals. The system and method can be used in detecting the activities of multiple individuals, which can be used in tracking recovery progress and/or detecting unpermitted activity (e.g., if a patient ignores bed rest requests). The hospitalization use case involves monitoring a plurality of patients. Each patient preferably uses at least one instance of the activity monitoring device and is monitored according to the described method. This monitoring can occur continuously while the patient is in the hospital so as to ensure all biomechanical signals are received, and can trigger various alerts as needed to the hospital staff if the patient is detected to be at high risk or behaving in a manner which could be problematic to him. Biomechanical analysis from a patient is preferably communicated to a remote monitoring system from which an administrator or worker or dispatcher can respond to the information. When applied in a multiple patient scenario, the method can additionally include detecting an event of at least one patient and triggering an alert to a remote monitoring system. The triggering event can be sent to a centralized system, or to specific staff on mobile units so as to enable faster response to the patient during the event. Delivering a health assessment in the context of multiple patients may include reporting on each patient's fulfillment of activity targets. An activity target could be a minimum amount of activity such as walking, motion, standing and the like. This may be used by a caregiver for understanding which patients are moving around better or not on any given day. This may be particularly useful in an elderly care scenario. Additionally, this system and method can be used to track the patients who should not be moving due to their current conditions. In this case, if patients have been detected to sit up in bed, get out of bed, or walk, an alert can be sent to a remote monitoring system.

In a similar use case, the system and method can be applied to monitoring quality of care within a population. The quality of care can be monitored within a hospital, across a subset of patients of a particular physician, for patients of an insurance provider, or for any suitable set of individuals. The quality of care use case preferably uses a combination of the above approaches to measure the health impact of an operation, medical device, pharmaceutical, treatment, doctor, and/or caretaker. The quality of care implementation involves the collection of biomechanical signals from a plurality of individuals. The nature of the individuals' conditions and of their recovery can be characterized through biomechanical signals and then compared to others with similar conditions or compared to a relevant healthy reference base average. The system and method could be used in helping patients understand the real-life impact of a particular operation vs. another operation. This can be similarly used to help in rating the quality of caretakers for particular conditions and symptoms. For example, for a particular type of injury different operations and doctors may have different levels of success. The system and method could be used in parameterizing such patterns. In another example, comparing the patient's biomechanical signals to a population data set, reference standard, or population that has been through a similar operation, may help the patient and provider determine the best treatment approach for the patient, given the patient's individual biomechanical signal patterns. This method can then be applied to help doctors, nurses, and other care practitioners improve the delivered quality of care to their patients.

### Other Variations

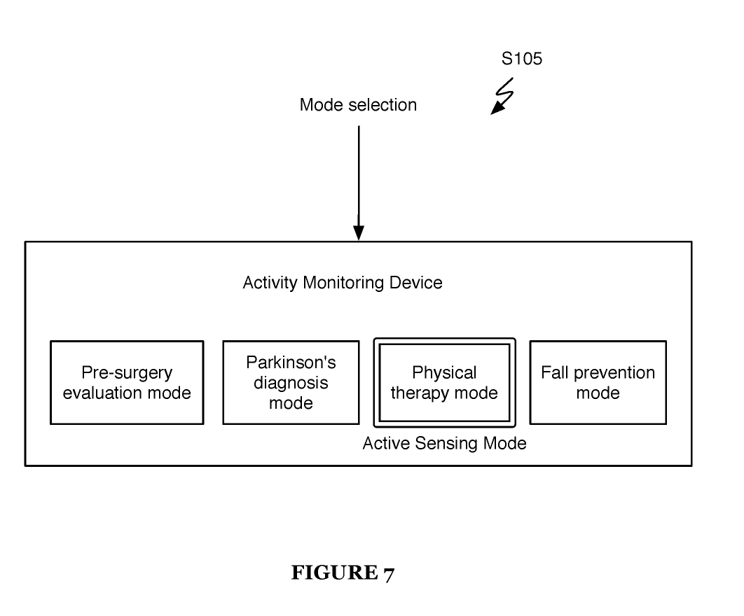

In one variation, the method can additionally include configuring the sensing mode of an activity monitoring device S105 as shown in FIG. 7, which functions to enable an activity monitoring device to be selectively used in different use cases. Configuring the sensing mode of an activity monitoring device can include setting the operating mode for different use cases such as a particular treatment administration use case, a particular diagnostics use case, an event detection use case, and/or any suitable use case. The sensing mode can alter the type of biomechanical signals to be generated and/or the delivered health assessment. The sensing mode can also determine the location where a sensor is to be placed on the body. For example, if the sensor mode is to detect shuffle gait, the application may guide the user to place the sensor on the foot. If the sensor mode is to detect upper body slouching, the system may guide the user to place the sensor on the clavicle. If the mode is chosen to measure knee range of motion, the user will be prompted to position a sensor above and below the knee.

In some instances, the sensor can also automatically detect the location where the sensor is being worn on the human body. This can automatically trigger a change to the sensing mode without the user needing to provide manual input as a form of simplified user experience, especially for elderly patients and patients suffering from motor control issues. Automatic location sensing can also help insure that the user placed the sensor in the right location for the right application sensing mode. For instance, if the application guides the user to put the sensor on the foot to detect shuffle gait, but the user accidentally places the sensor on the upper clavicle, the application can prompt the user to correct the mistake or ask the user if they want to change to a different application based on the clavicle sensor location.

In one variation, the method may be used exclusively for a single sensing mode, in which case there may be no changing or dynamic determination of a sensing mode. However, even in the case where a single biomechanical signal is to be generated there may be an active mode and an inactive mode to only collect relevant biomechanical signal information when the associated activity is being performed. In another variation, the activity monitoring device may have multiple applications within the health space, and a doctor or other administrator could update the settings of the activity monitoring device so that the device can be used in different situations. This can make the activity monitoring device a flexible tool that is useful in a variety of situations. Additionally, the activity monitoring device can be updated with new use case sensing modes as they are developed or improved.

Determining a sensing mode can be dynamically selected. The kinematic data and/or other signals may be used in activating a sensing mode. For example, one implementation may automatically detect walking biomechanical signals and dynamically change to a walking mode, automatically change to a running mode when the patient starts jogging, a repose mode when the patient is determined to be lying down, and return to an inactive mode when not worn or when the patient is not active. Additionally, the sensing mode may change depending on the location it is being worn. The system and method detects that the device is worn on the foot, it may switch (or prompt the user) to enter into a foot biomechanics sensing mode, and if the device detects that is being worn on the pelvis, it may automatically change to a pelvis biomechanical mode for walking if the user was walking, and a pelvis biomechanical mode for running if the user was detected to run.

Determining a sensing mode may alternatively be remotely selected. Remote selection of a sensing mode may be initiated by a user such as a doctor, a trainer, or the patient. The most applicable sensing mode may also be recommended to the doctor, trainer, or patient before the user makes any changes, so as to ensure the correct data collection for the specific use case. An administration interface preferably enables an individual to activate a sensing mode. For example, prior to doing a fitness test on a treadmill, a doctor may activate a walking mode for a patient. The results of the fitness test can be recorded and used in delivering a health assessment for that particular activity. In another variation, the sensing mode can be triggered by any suitable signal. For example, a patient recovering in a hospital may trigger a walk-sensing mode when the patient leaves the vicinity of his hospital bed.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A method for addressing patient health through monitoring patient movement comprising:
in association with at least one treatment stage, collecting kinematic data from at least one inertial measurement unit of an activity monitoring system, wherein the activity monitoring system is coupled to a subject;
generating a set of biomechanical signals from the kinematic data wherein the set of biomechanical signals characterize at least one biomechanical property;
updating a mobility quality score of the subject based on the set of biomechanical signals; and
delivering a health assessment.

2. The method of claim 1, further comprising tracking administration of a treatment; and wherein the health assessment is an analysis of the treatment based in part on the motion quality in comparison to the administration of the treatment.

3. The method of claim 2, wherein the treatment is a set of physical therapy sessions, and wherein the assessment indicates mobility quality relative to tracked physical therapy sessions.

4. The method of claim 2, wherein the treatment is a set of physical therapy sessions, and wherein the health assessment includes a physical therapy treatment recommendation.

5. The method of claim 2, wherein the treatment is administration of a drug, and wherein the health assessment includes a drug treatment recommendation with recommended drug dosage and timing.

6. The method of claim 5, wherein the drug is a pain medicine.

7. The method of claim 2, wherein the treatment is usage of a medical device.

8. The method of claim 1, wherein collecting the kinematic data and generating the set of biomechanical signals are performed during the recovery stage of a treatment; and wherein the assessment is a recovery report.

9. The method of claim 1, wherein the treatment stage is a treatment analysis stage, and wherein the assessment includes a metric for predicted treatment impact.

10. The method of claim 1, wherein the treatment stage is a post-surgery analysis stage.

11. The method of claim 10, wherein delivering the health assessment comprises comparing current motion quality score from a current post-surgery stage to a motion quality score from a pre-surgery stage.

12. The method of claim 1, wherein the treatment stage is a diagnosis stage; further comprising directing actions by a patient; wherein biomechanical signals are generated during each directed action, wherein the health assessment is a motion disorder diagnosis report.

13. The method of claim 1, wherein updating the mobility quality score comprises detecting an event risk scenario through the biomechanical signals; and wherein delivering the health assessment comprises triggering an alert in response to the event risk scenario.

14. The method of claim 1, wherein generating the set of biomechanical signals comprises generating a set of stride-based biomechanical signals comprising segmenting kinematic data by steps and for at least a subset of the stride-based biomechanical signals generating a biomechanical signal based on step biomechanical properties.

15. The method of claim 14, wherein generating the set of stride-based biomechanical signals comprises generating a step consistency biomechanical signal.

16. The method of claim 1, wherein kinematic data is collected from at least two inertial measurement units; and further comprising analyzing relative displacements between locations of the two inertial measurement units and determining a range of motion estimate; and wherein mobility quality is based in part on range of motion.

17. A method for addressing patient health through monitoring patient movement comprising:
monitoring mobility quality of a plurality of patients, which comprises for each patient:
collecting kinematic data from at least one inertial measurement unit of an activity monitoring system, wherein the activity monitoring system is coupled to a subject,
generating a set of biomechanical signals from the kinematic data wherein the set of biomechanical signals characterize at least one biomechanical property,
updating a mobility quality score of the patient based on the set of biomechanical signals, and
delivering a health assessment to an administrator system.

18. The method of claim 17, further comprising detecting an event of at least one patient, and triggering an alert to a remote monitoring system.

19. The method of claim 17, wherein delivering a health assessment to an administrator system can comprise reporting on a patients fulfillment of an activity target.

20. A system comprising:
an activity monitoring device that comprises at least one inertial measurement unit, wherein the activity monitoring device is configured to collect kinematic data during at least one stage of a treatment; and
a processor, wherein the processor is configured to
generate a set of biomechanical signals from the kinematic data, wherein the set of biomechanical signals characterize at least one biomechanical property,
update a mobility score of a patient based on the set of biomechanical signals, and
deliver a health assessment.
